(19) United States
(12) Patent Application Publication
(10) Pub. No.: US 2026/0123951 A1
(43) Pub. Date: May 7, 2026

(54) DEVICES, SYSTEMS, AND METHODS FOR SEPARATING TISSUE

(71) Applicant: BOSTON SCIENTIFIC SCIMED, INC., Maple Grove, MN (US)

(72) Inventors: Alexander R. Cohen, Austin, TX (US); Ryan Davis Gilkey, Austin, TX (US)

(73) Assignee: BOSTON SCIENTIFIC SCIMED, INC., Maple Grove, MN (US)

(21) Appl. No.: 19/359,364

(22) Filed: Oct. 15, 2025

Related U.S. Application Data
(60) Provisional application No. 63/709,196, filed on Oct. 18, 2024.

(51) Int. Cl. A61B 17/32; A61B 17/00

(52) U.S. Cl. CPC A61B 17/320016; A61B 2017/00367; A61B 2017/00867; A61B 2017/320044

(57) ABSTRACT

Devices, systems, and methods for separating tissue using blunt tissue-separators. The blunt tissue separators may include two or more elongated elements with distal ends adjacent each other when inserted into tissue to be separated. The distal ends are moved apart to separate the tissue, such as by snapping the tissue without cutting the tissue. The distal ends of the tissue separators may be moved proximally in addition to laterally to pull apart tissue as well as to further advance the device into tissue.

### CROSS-REFERENCE TO RELATED APPLICATIONS

This application claims the benefit of priority under 35 U.S.C. § 119 to U.S. Provisional Application No. 63/709,196, filed Oct. 18, 2025, the entire disclosure of which is hereby incorporated by reference herein for all purposes.

### FIELD

The present disclosure relates generally to the field of medical devices, systems, and methods for separating tissue. More particularly, the present disclosure relates to medical devices, systems, and methods using a blunt device with a pair of separators configured to mechanically separate or dissect tissue, such as tissue which is readily separable without a sharp cutting instrument.

### BACKGROUND

Various devices, assemblies, systems, methods, techniques, etc., exist for cutting, dissecting, resecting, excising, etc., anatomical structures (e.g., biological tissue, including lesions, etc.). The configurations of the cutting device (which may include a blade, needle, laser, or other structure capable of cutting anatomical structures) may be selected based on the particular procedure to be performed. The procedures may be performed using open surgery (accessing the interior of a patient's body by cutting open the body) or minimally invasive surgery (e.g., percutaneously, laparoscopically, endoscopically, etc.). One endoscopic procedure is third space endoscopy (also known as submucosal endoscopy), which may access deeper layers of tissue within the body (e.g., the gastrointestinal (GI) tract) by tunneling into the tissue, such as between structurally differentiated layers of tissue. For instance, in the GI system, tunneling may be performed in the submucosal space with an endoscope, without compromising the integrity of the overlying mucosa. Typically, a fluid (e.g., water, saline, air, etc.) is injected at the treatment site to elevate/lift the tissue (to create a “bleb”) to facilitate cutting of the tissue, such as with a knife. For instance, a lifting agent may be injected into the submucosal layer to separate the mucosal layer from the muscularis layer. A sharp cutting instrument such as a blade, a knife, an electrosurgical knife, etc., may then be used to cut through the submucosa. Typically, the cutting instrument is used to make multiple small cuts, typically sequentially, to create a desired separation of tissue gradually, such as to reduce the risk of creating too large a cut in the tissue. Safer and more efficient devices, systems, methods, etc., such as using blunt instruments, would be welcome in the field.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a device for separating tissue includes a first tissue separating member and a second tissue separating member which move apart from each other to separate tissue. In some aspects, the tissue separating members each have a blunt distal end so that the device may be considered a blunt tissue separator. In some aspects, the tissue separating members are configured to have an increased range of motion relative to tissue separating members of prior devices. In some aspects, the tissue separating members achiever a greater amount of tissue separation than prior devices, such as by the unique movements of the tissue separating members.

In some aspects, the device for separating tissue includes an elongate member having a proximal end and a distal end; a first tissue-separating member having a proximal end operably coupled with the distal end of the elongate member, and a distal end extending distally from the elongate member; a second tissue-separating member having a proximal end operably coupled with the distal end of the elongate member, and a distal end extending distally from the elongate member; a first tissue-separator actuator having a distal end operably coupled with the distal end of the first tissue-separating member; and a second tissue-separator actuator having a distal end operably coupled with the distal end of the first tissue-separating member. In some aspects, the first tissue-separator actuator and the second tissue-separator actuator are shiftable to shift the first tissue-separating member and the second tissue-separating member between a first configuration, in which the distal ends of the first tissue-separating member and the second tissue-separating member are adjacent each other, and a second configuration, in which the distal ends of the first tissue-separating member and the second tissue-separating member are spaced apart from each other.

In some aspects, when the first tissue-separating member and the second tissue-separating member are in the first configuration, the distal ends of the first tissue-separating member and the second tissue-separating member are at a first axial distance from the proximal ends of the first tissue-separating member and the second tissue-separating member, and when the first tissue-separating member and the second tissue-separating member are in the second configuration, the distal ends of the first actuator and the second actuator are at a second axial distance from the proximal ends of the first tissue-separating member and the second tissue-separating member, the second axial distance being less than the first axial distance.

In some aspects, the first tissue-separator actuator and the second tissue-separator actuator are shiftable to shift the distal ends of the first tissue-separating member and the second tissue-separating member from the first configuration adjacent each other and at a first axial distance from the proximal ends of the first tissue-separating member and the second tissue-separating member to a configuration in which the distal ends of the first tissue-separator actuator and the second tissue-separator actuator are at a second axial distance from the proximal ends of the first tissue-separating member and the second tissue-separating member, the second axial distance being less than the first axial distance.

In some aspects, the first tissue-separating member and the second tissue-separating member bend away from each other when shifting from the first position to the second position.

In some aspects, shifting of the first tissue-separating member and the second tissue-separating member to the second configuration moves tissue contacted by the first tissue-separating member and the second tissue-separating member apart as well as proximally. In some aspects, the tissue is moved apart laterally with respect to a longitudinal axis of the device.

In some aspects, the device further includes a control handle and an actuator extending from a proximal end operably associated with the control handle distally to a distal end operably coupled with the first tissue-separator actuator and the second tissue-separator actuator to axially shift the first tissue-separator actuator and the second tissue-separator actuator to shift the first tissue-separating member and the second tissue-separating member between the first configuration and the second configuration.

In some aspects, in the first configuration, the first tissue-separating member is adjacent the first tissue-separator actuator and the second tissue-separating member is adjacent the second tissue-separator actuator; and in the second configuration, the first tissue-separating member is spaced apart from the first tissue-separator actuator and the second tissue-separating member is spaced apart from the second tissue separator actuator.

In some aspects, the first tissue-separating member includes a pair of tissue-separating members, and the second tissue-separating member includes a pair of tissue-separating members.

In some aspects, the first tissue-separating member and the second tissue-separating member are formed from shape memory materials.

In accordance with various principles of the present disclosure, the device for separating tissue includes a first tissue separator having a proximal end and a distal end and comprising a flexible portion and an actuator portion extending between the proximal end and the distal end; and a second tissue separator having a proximal end and a distal end and comprising a flexible portion and an actuator portion extending between the proximal end and the distal end. In some aspects, the first tissue separator and the second tissue separator are shiftable between a first configuration in which the distal ends of the first tissue separator and the second tissue separator are adjacent each other, and a second configuration in which the distal ends of the first tissue separator and the second tissue separator are apart from each other; and when the first tissue separator and the second tissue separator are shifted to the second configuration, a distance between the flexible portion and the actuator portion of each of the first tissue separator and the second tissue separator increases.

In some aspects, shifting of the first tissue separator and the second tissue separator from the first configuration to the second configuration increases a tissue separating area defined by the first tissue separator and the second tissue separator.

In some aspects, shifting of the first tissue separator and the second tissue separator toward the second configuration moves the distal ends of the first tissue separator and the second tissue separator proximally.

In some aspects, wherein shifting of the first tissue separator and the second tissue separator toward the second configuration causes the flexible portions of the first tissue separator and the second tissue separator to bend away from each other. In some aspects, shifting of the first tissue separator and the second tissue separator causes the flexible portions of the first tissue separator and the second tissue separator to flex relative to the actuator portions. In some aspects, each of the flexible portion and the actuator portion of the first tissue separator and the second tissue separator has a proximal end and a distal end; the actuator portion of the first tissue separator has a distal end coupled to distal end of the flexible portion of the first tissue separator; the actuator portion of the second tissue separator has a distal end coupled to a distal end of the flexible portion of the second tissue separator; and the device further includes an actuator coupled to proximal ends of the actuator portions of the first tissue separator and the second tissue separator to proximally pull the actuator portions to pull the distal ends of the flexible portions proximally to shift the first tissue separator and the second tissue separator toward the second configuration.

In accordance with various principles of the present disclosure, a device for separating tissue includes an elongate member having a proximal end and a distal end; a first tissue-separating member having a proximal end operably coupled with the distal end of the elongate member, and a distal end extending distally from the elongate member; a second tissue-separating member having a proximal end operably coupled with the distal end of the elongate member, and a distal end extending distally from the elongate member. In some aspects, the proximal ends of the tissue separating members are operably associated with gears engaged with each other to effect pivoting of the tissue-separating members. In some aspects, the gears are formed along the proximal ends of the tissue separating members. In some aspects, an actuator is operably associated with one of the tissue separating members to move the one of the tissue separating members. In some aspects, movement of one of the tissue separating members causes movement of the other of the tissue separating members. In some aspects, movement of one of the tissue separating members causes movement of a first gear associated with the one of the tissue separating members, and movement of the first gear causes movement of a second gear associated with the other of the tissue separating members to cause movement of the other of the tissue separating members.

In accordance with various principles of the present disclosure, a method of separating tissue includes advancing a tissue separator device to tissue to be separated, the tissue separator device having a first tissue-separating member with a proximal end and a distal end, and a second tissue-separating member with a proximal end and a distal end; inserting the tissue separator device into tissue in a first configuration with the distal ends of the first tissue-separating member and the second tissue-separating member adjacent each other; and shifting the tissue separator device towards a second configuration to move the distal ends of the first tissue-separating member and the second tissue-separating member laterally apart from each other as well as proximally to move the tissue to be separated apart (e.g., laterally) as well as proximally.

In some aspects, the method further includes increasing the surface area of the first tissue-separating member and the second tissue-separating member upon shifting the tissue separator device towards the second configuration.

In some aspects, the method further includes causing a flexible portion of the first tissue-separating member to bend outwardly away from a flexible portion of the second tissue-separating member when shifting the tissue separator device towards the second configuration.

In some aspects, the method further includes pulling an actuator proximally to pull the distal ends of the first tissue-separating member and the second tissue-separating member proximally. In some aspects, pulling the distal ends of the first tissue-separating member and the second tissue-separating member proximally causes the distal ends of the first tissue-separating member and the second tissue-separating member to move apart from each other.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue separating system formed in accordance with aspects of the present disclosure, including a detail view of an example of an embodiment of a tissue separator device thereof formed in accordance with various principles of the present disclosure and in a first configuration.

FIG. 2 illustrates a perspective view of a second configuration of the tissue separator device illustrated in FIG. 1.

FIG. 3 illustrates a cross-sectional view along line III-III of FIG. 1.

FIG. 4 illustrates a perspective view of an example of an embodiment of a tissue separating system formed in accordance with aspects of the present disclosure, including a detail view of an example of an embodiment of a tissue separator device formed in accordance with various principles of the present disclosure and in a first configuration.

FIG. 5 illustrates an elevational view of a first configuration of the tissue separator device illustrated in FIG. 4.

FIG. 6 illustrates an elevational view of a second configuration of the tissue separator device illustrated in FIG. 4.

FIG. 7 illustrates a perspective view of an example of an embodiment of a tissue separator device formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Devices, systems, and methods for separating tissue formed in accordance with various principles of the present disclosure, such as described herein, include generally elongated tissue separators with distal ends movable from a first configuration, in which the distal ends are adjacent to each other, to a second configuration, in which the distal ends are spaced apart from each other (a greater distance than in the first configuration). The tissue separators may be inserted, in the first configuration, into an initial incision in tissue. As the tissue separators move apart to the second configuration, each tissue separator moves tissue away from the other tissue separator. Specifically, each tissue separator has an inner side, facing the other tissue separator, and an outer side facing away from the other tissue separator. Movement of one of the tissue separators causes movement of tissue on the outer side of each of the tissue separators to move away from the other of the tissue separators.

In some aspects, the movement of the tissue separators is akin to a breaststroke swimming motion in which the tissue separators are advanced distally into tissue, and then moved laterally apart and proximally, pushing and/or pulling tissue both apart as well as proximally, thus further distally advancing the tissue separators into the tissue. In some aspects, the tissue is moved apart laterally. In some aspects, each tissue separator is generally elongated with a blunt distal end which does not cut tissue, but, instead, advances into tissue to separate the tissue.

Various embodiments of devices, systems, and methods for separating tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

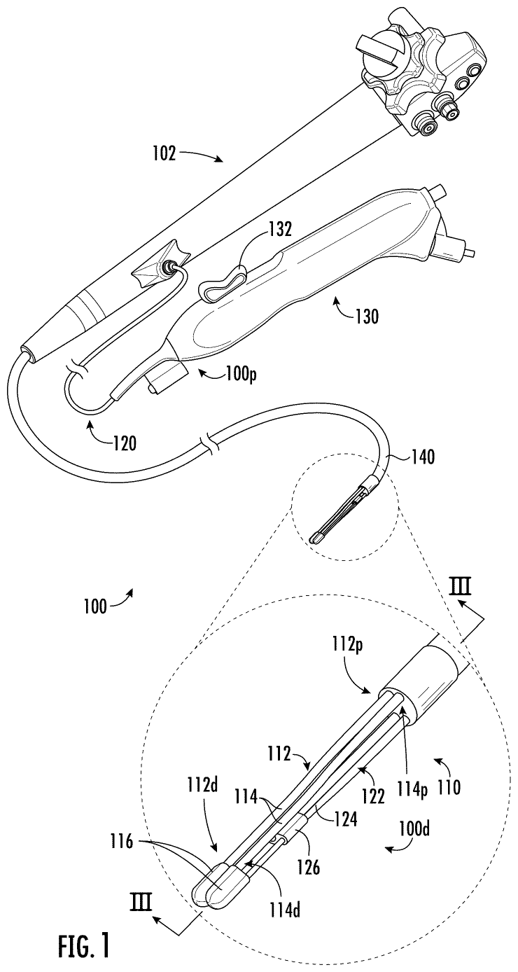

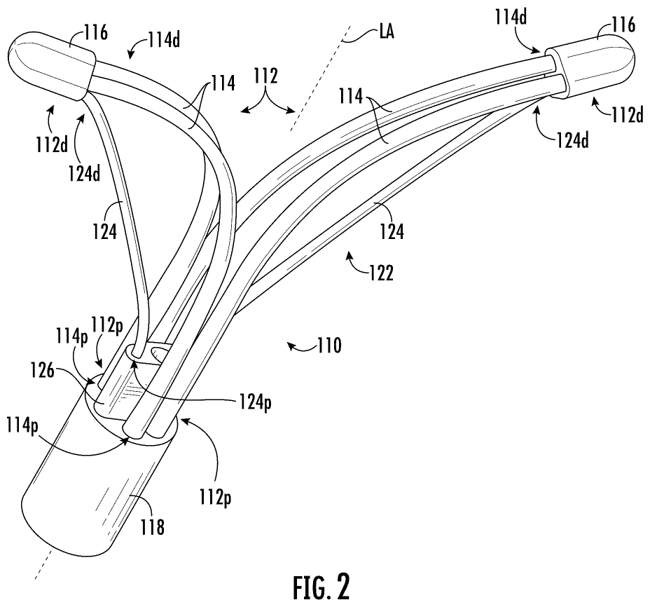

Turning now to the drawings, an example of an embodiment of a tissue-separating system 100 formed in accordance with various principles of the present disclosure is illustrated in FIG. 1. The illustrated tissue-separating system 100 includes a tissue separator device 110 along a distal end 100d of the tissue-separating system 100. In accordance with various principles of the present disclosure, the tissue separator device 110 includes at least two tissue separators 112 movable between a delivery configuration and a tissue-separating configuration. In the delivery configuration, the tissue separators 112 extend generally alongside each other, such as illustrated in FIG. 1. In some aspects, the tissue separators 112 may be in a relatively compact configuration (at least relative to other configurations of the tissue separators 112) when in the delivery configuration to facilitate delivery within a patient's body (e.g., transluminally). In some aspects, the tissue separators 112 may be adjacent each other, when in the delivery configuration, to facilitate insertion into tissue (e.g., through an incision made by another device, such as a sharper cutting instrument). In some aspects, the tissue separators 112 are inserted together into tissue, such as without intentionally extending tissue between the adjacent tissue separators 112. In the tissue-separating configuration, such as illustrated in FIG. 2, the tissue separators 112 are moved apart from each other, such as to move apart tissue surrounding the tissue separators 112 when the tissue separators 112 are in the delivery configuration. It will be appreciated that separation of tissue or moving apart tissue as described herein, such as upon moving apart the tissue separators 112 of a tissue separator device 110 formed in accordance with various principles of the present disclosure, may be generally in a direction laterally with respect to the longitudinal axis LA of the tissue separator device 110, including at an angle greater than 0° and less than 180° with respect to the longitudinal axis LA.

An actuator 120, operable to actuate the two tissue separators 112 of the tissue separator device 110, extends proximally from the tissue separator device 110 to a control handle 130 configured to operate the actuator 120. It will be appreciated that the tissue-separating device and the control handle 130 are not necessarily drawn on the same scale in FIG. 1. In some aspects, at least a distal portion of the actuator 120 may be considered a component of the tissue separator device 110. In some aspects, a sheath 140 (e.g., tubular element, shaft, covering, etc.) may be provided over the actuator 120, between the tissue separator device 110 and the control handle 130. It will be appreciated that at least the actuator 120 and the sheath 140 may be formed as elongate flexible elements with sufficient flexibility to be navigated within a patient's body, such as through tortuous passages within the patient's body, for use in endoluminal, such as endoscopic, procedures. In some aspects, the tissue separator device 110 and the actuator 120 are advanced through a port of an medical scope 102 for delivery to a treatment site through a working channel of the medical scope, such as in a manner known to those of ordinary skill in the art and as illustrated schematically in FIG. 1.

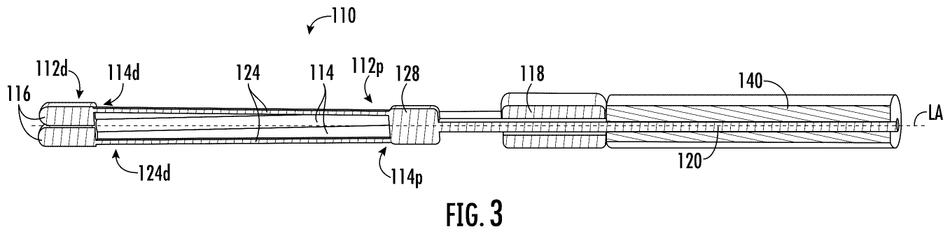

In the example of an embodiment illustrated in FIG. 1, FIG. 2, and FIG. 3, each of the tissue separators 112 of the tissue separator device 110 includes at least one elongate tissue-separating member 114, and, in the illustrated embodiment, at least two elongate tissue-separating member 114. In accordance with various principles of the present disclosure, the tissue separators 112 may be shifted from a first configuration in which the distal ends 112d of the tissue separators 112 are adjacent each other (such as for insertion into tissue), such as illustrated in FIG. 1, into a second configuration which may be referenced as a tissue-separating configuration, such as illustrated in FIG. 2. In some aspects, the tissue separators 112 move towards a tissue-separating configuration by bending the elongate tissue-separating members 114 thereof laterally away from the longitudinal axis LA of the tissue separator device 110. It will be appreciated that terms such as flex, bend, bow, arc, curve, etc., including other grammatical forms thereof, are usable interchangeably herein without intent to limit. In some aspects, the bending is substantially arcuate, with convex curvatures of the elongate tissue-separating members 114 facing each other. In some aspects, the distal ends 114d of the elongate tissue-separating members 114 are brought closer to the proximal ends 114p thereof as the tissue separators 112 are shifted to the tissue-separating configuration. As may be appreciated, bowing of the elongate tissue-separating members 114 of the tissue separators 112 as the tissue separators 112 are shifted to a tissue-separating configuration moves the distal tips 116 apart from each other faster than if the elongate tissue-separating members 114 remained substantially straight as they shifted apart from one another. As such, a tissue separator device 110 formed in accordance with various principles of the present disclosure may be used for quick dissection of tissue, and may safely be used to pull apart tissue in a controlled yet quick manner (e.g., relative to prior art devices which may utilize relatively rigid and/or straight elongate elements).

In some aspects, one or both of the tissue separators 112 include a blunt distal tip 116 along a distal end 112d of the tissue separator 112. It will be appreciated that the distal tip 116 may be formed by rounding the distalmost ends of the elongate tissue-separating member 114 and/or may be formed separately from the elongate tissue-separating members 114 and coupled thereto (e.g., welded, adhered, friction fit, etc. together). The blunt distal tip 116 may be rounded, curved, convex, or otherwise shaped to facilitate insertion of the tissue separators 112 into tissue to be separated without inadvertently damaging the tissue (e.g., cutting blood vessels, connective tissue, etc., not intended to be cut). As the tissue separators 112 move to the tissue-separating configuration, the distal tips 116 of the tissue separators 112 are moved apart along with the elongate tissue-separating members 114 to separate tissue.

In the example of an embodiment of a tissue separator device 110 illustrated in FIG. 1, FIG. 2, and FIG. 3, a tissue-separator actuator 122 is operably associated with the tissue separator 112. More particularly, in the illustrated example of an embodiment, a tissue-separator actuator 122 is associated with each tissue separator 112. In the example of an embodiment illustrated in FIG. 1, FIG. 2, and FIG. 3, each of the tissue separator actuators 122 includes at least one elongate actuator member 124 extending along the tissue separators 112 to cause the elongate tissue-separating members 114 thereof to flex and shift into a tissue-separating configuration. More particularly, in the example of an embodiment illustrated in FIG. 1, FIG. 2, and FIG. 3, the distal end 124d of an elongate actuator member 124 is coupled to a distal end 114d of an associated elongate tissue-separating member 114, and the proximal end 124p of the elongate actuator member 124 is coupled to the actuator 120. As may be appreciated proximal movement of the actuator 120 moves the elongate actuator members 124 of the tissue-separator actuators 122 proximally. In some aspects, proximal movement of the actuator 120 pulls the distal ends 114d of the elongate tissue-separating members 114 of the tissue separators 112 proximally towards the proximal ends 114p of the elongate tissue-separating members 114. In some aspects, the distal ends 114d of the elongate tissue-separating members 114 of the tissue separators 112 are at a first axial distance from the proximal ends 114p of the elongate tissue-separating members 114 in a first configuration, and are shifted proximally, upon pulling the actuator 120 proximally, to a second configuration in which the distal ends 114d of the elongate tissue-separating members 114 of the tissue separators 112 are at a second axial distance, greater than the first axial distance, from the proximal ends 114p of the elongate tissue-separating members 114. In some aspects, proximal pulling of the actuator 120 causes the tissue separators 112 to bow laterally outwardly and away from each other. In some aspects, the proximal end 124p of one or both of the elongate actuator members 124 is operably coupled with the actuator 120 via a connector 126.

In some aspects, an elongate actuator member 124 extends along the elongate tissue-separating member 114 of each of the tissue separators 112, with the proximal ends 124p of the elongate actuator members 124 coupled to a common connector 126 operably coupled with the actuator 120. In some aspects, the tissue-separator actuator 122 and the connector 126 may be considered to be a component of the actuator 120, and/or these components may be considered components of the tissue separator device 110. In some aspects, the elongate tissue-separating members 114 (e.g., the proximal end 114p thereof) are operably coupled with a collar 118, and the actuator 120 axially translatably extends through the collar 118 to be operably coupled with the connector 126 to actuate the tissue-separator actuators 122, such as may be appreciated with reference to the cross-sectional view (along line III-III of FIG. 1) illustrated in FIG. 3. In some aspects, the collar 118 is operably coupled with the distal end 140d of the sheath 140. In some aspects, a user-engaged actuator 132, such as a slider, is provided along the control handle 130, as illustrated in FIG. 1, and is operably coupled with the actuator 120 to axially translate the actuator 120 to shift the configuration of the tissue separator device 110.

As may be appreciated, and as noted above, proximal and laterally outward movement of the tissue separators 112 into the tissue-separating configuration not only separates tissue, but also move tissue proximally. Moreover, by moving the tissue proximally, the tissue separators 112 and the tissue separator device 110 are advanced further distally and deeper into tissue to be separated. The movement of the tissue separators 112 of a tissue separator device 110 formed in accordance with various principles of the present disclosure thus achieves faster dissection of tissue than achievable by prior devices which remain generally linear and/or simply pivot open about a proximal pivot point. Moreover, as may be appreciated upon comparison of the configuration of the tissue separators 112 illustrated in FIG. 1 with the configuration of the tissue separators 112 illustrated in FIG. 2, in some aspects, the overall dimensions of the tissue separators 112 increase as the tissue separators 112 shift from the delivery configuration to a tissue-separating configuration. More particularly, in some aspects, the elongate actuator members 124 are not as flexible and/or do not bend with the same curvature to which the elongate tissue-separating members 114 bend. Instead, as illustrated in FIG. 2, the elongate tissue-separating member 114 bend to a greater extent (e.g., have a smaller radius of curvature) than the elongate actuator members 124 in a tissue-separating configuration. The elongate actuator members 124 may thus be considered to move proximally away from the elongate tissue-separating members 114 as the tissue separators 112 move apart. Such movement of the elongate actuator members 124 draws tissue proximally. Moreover, as the distance between the elongate tissue-separating members 114 and the elongate actuator members 124 increases as the tissue separators 112 move further apart, tissue is further spread apart (e.g., moved laterally apart) by the elongate tissue-separating members 114 and the elongate actuator members 124. For instance, if each of the elongate tissue-separating members 114 is considered to have an inner side which faces another elongate tissue-separating member 114 (directed towards the longitudinal axis LA of the tissue separator device 110), and an outer side which faces away from the other elongated tissue-separating member 114 (and away from the longitudinal axis LA) and along which an elongate actuator member 124 extends, then as a tissue separator 112 shifts from the delivery configuration to a tissue-separating configuration, the elongate actuator member 124 moves away from the inner side of the elongate tissue-separating member 114 and increases the dimension of the tissue separator 112 in a direction away from the longitudinal axis LA, spreading tissue as such dimension increases.

In use, a tissue separator device 110 formed in accordance with various principles of the present disclosure can be shifted from a delivery configuration (such as illustrated in FIG. 1) to a tissue-separating configuration (such as illustrated in FIG. 2) by pulling proximally on the actuator 120 to pull the elongate actuator members 124 proximally (as may be appreciated with reference to FIG. 3) to pull the distal ends 114d of the elongate tissue-separating members 114 laterally outwardly as well as proximally. Tissue may thereby be pulled apart safely (with little to no risk of inadvertently cutting blood vessels or other anatomical structures not intended to be cut) in a controlled manner. For instance, movement of the elongate tissue-separating member 114 is controllable to exert selected and/or different magnitude and/or direction of forces on tissue, in contrast with simply pushing into tissue (e.g., with a medical scope). In some aspects, the tissue separator device 110 can be used to grasp tissue, such as to manipulate tissue. For instance, in some procedures (e.g., third space endoscopy), the tissue separator device 110 may be used to grasp and pull and snap tissue to separate tissue. Various additional devices may be used in conjunction with the tissue separator device 110. For instance, a cutting instrument such as a blade, a knife, an electrocautery knife, scissors, etc., may be used to create an initial incision into which the distal end 110d of the tissue separator device 110 is inserted in a delivery configuration. Additionally or alternatively, a device such as an cauterization or other coagulation device may be used to address blood vessels which cannot be cut with the spreading action of the tissue separator device 110.

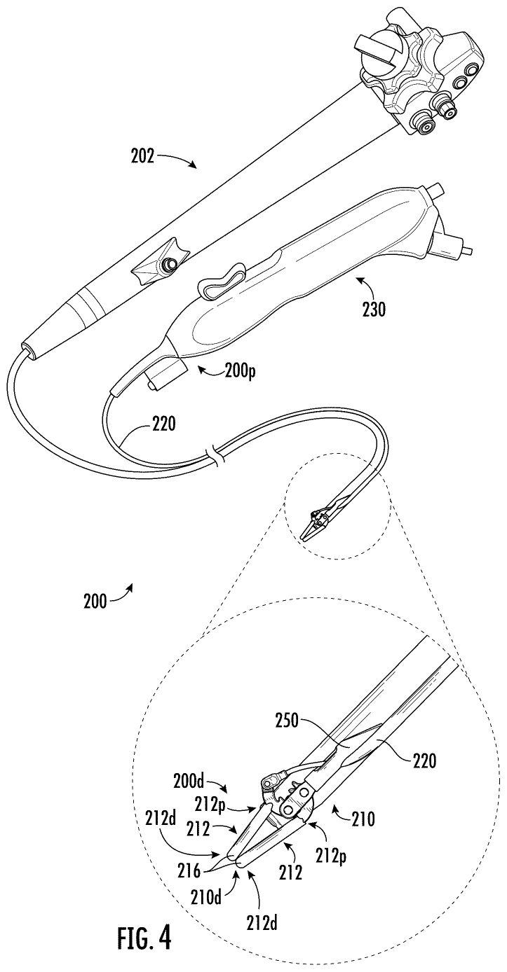

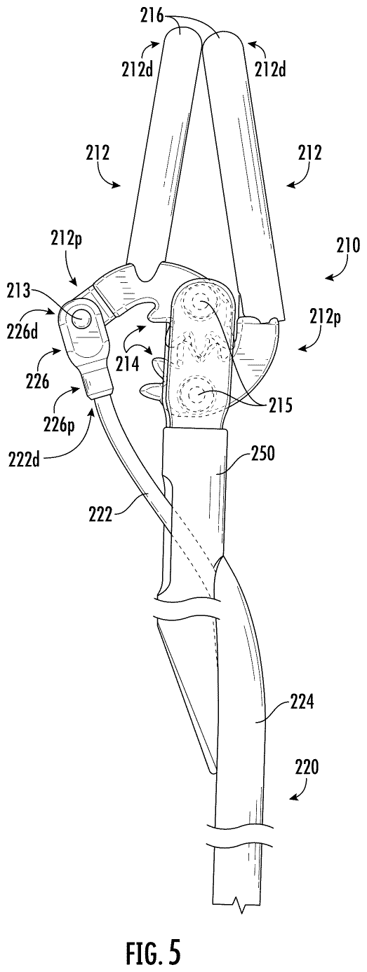

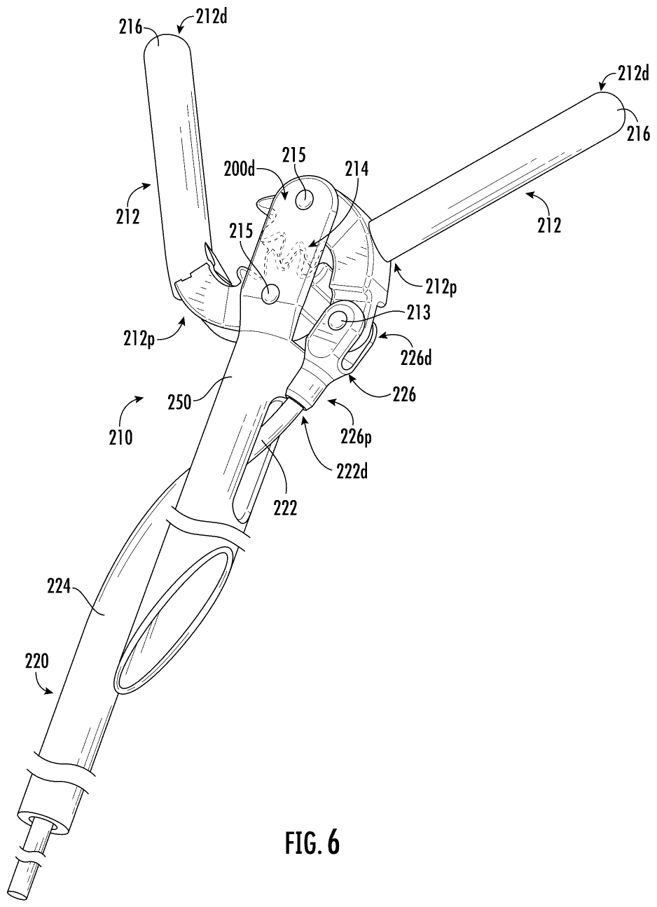

It will be appreciated that various principles of the present disclosure may be applied in other forms and configurations of tissue separator devices. Another example of an embodiment of a tissue-separating system 200 formed in accordance with various principles of the present disclosure is illustrated in FIG. 4, FIG. 5, and FIG. 6. Similar to the tissue-separating system 100 illustrated in FIG. 1, the tissue-separating system 100 of FIG. 4 includes a tissue separator device 210 along a distal end 200d of the tissue-separating system 200, and an actuator 220 extending proximally from the tissue separator device 210 to a control handle 230 at the proximal end 200p of the tissue-separating system 200. In some aspects, a mount 250 facilitates operable coupling of the tissue separator device 210 and the actuator 220. The mount 250 may be in any desired configuration, limited to the distal end 200d of the tissue-separating system 200, or extending further proximally than illustrated, the present disclosure not being limited in this regard. The tissue separator device 210 of the tissue-separating system 200 illustrated in FIGS. 4-6 may be configured to be delivered alongside and/or over the portion of a medical scope 202 which is inserted into a patient's body (a flexible elongate member such as an insertion tube), such as illustrated in FIG. 4.

Similar to the tissue separator device 110 illustrated in FIGS. 1-3, the tissue separator device 210 illustrated in FIGS. 4-6 includes at least two tissue separators 212 movable between a delivery configuration (such as illustrated in FIG. 4 and FIG. 5) and a tissue-separating configuration (such as illustrated in FIG. 6). Also similar to the tissue separators 112 illustrated in FIGS. 1-3, one or both of the tissue separators 212 of the tissue separator device 210 illustrated in FIGS. 4-6 include a blunt distal tip 216 along a distal end 212d of the tissue separator 212. It will be appreciated that the distal tip 216 may be formed by rounding the distalmost ends of the tissue separators 212 and/or may be formed separately from the tissue separators 212 and coupled thereto (e.g., welded, adhered, friction fit, etc. together). The blunt distal tips 216 may be rounded, curved, convex, or otherwise shaped to facilitate insertion of the tissue separators 212 into tissue to be separated without inadvertently damaging the tissue (e.g., cutting blood vessels, connective tissue, etc., not intended to be cut).

In the delivery configuration, the distal ends 212d of the tissue separators 212 extend generally alongside each other, such as illustrated in FIG. 4 and FIG. 5. In some aspects, in contrast with the tissue separators 112 illustrated in FIGS. 1-3, the tissue separators 212 may be configured and/or positioned with the distal ends 212d thereof closer to each other than the proximal ends 212p thereof. In other words, the distal ends 212d of the tissue separators 212 may be adjacent to each other, whereas the proximal ends 212p of the tissue separators 112 may be spaced apart from each other. In such configuration, the tissue separators 212 may be considered to form a tapered distal end 210d of the tissue separator device 210 which may facilitate insertion of at least the tissue separators 212 into tissue, such as into an incision made by another device, such as a sharper cutting instrument, such as described.

Similar to the tissue separator device 110 illustrated in FIGS. 1-3, the tissue separators 212 of the tissue separator device 210 illustrated in FIGS. 4-6 are moved apart from each other, such as to move apart tissue surrounding the tissue separators 212 when the tissue separators 212 are in the delivery configuration. However, the manner in which the tissue separators 212 of the tissue separator device 210 illustrated in FIGS. 4-6 shift between a delivery configuration and a tissue-separating configuration differs from the manner in which the tissue separators 112 of the tissue separator device 110 illustrated in FIGS. 1-3 shift between a delivery configuration and a tissue-separating configuration. In accordance with various principles of the present disclosure, the proximal ends 212p of the tissue separators 212 of the tissue separator device 210 illustrated in FIGS. 4-6 may be pivotably coupled together to pivot with respect to each other between a delivery configuration (such as illustrated in FIG. 4 and FIG. 5) and a tissue-separating configuration (such as illustrated in FIG. 6). Further in accordance with various principles of the present disclosure, the proximal ends 212p of the tissue separators 212 are operably associated with gears 214 engaged with each other to effect pivoting of the tissue separators 212 in a manner which achieves a mechanical advantage over pivotable coupling about a pivot pin or hinge, such as in prior art devices. In some aspects, the gears 214 are formed along the proximal ends 212p of the tissue separators 212, such as to be formed as a part of the proximal ends 212p of the tissue separators 212. In some aspects, the gears 214 are pivotably coupled to a mount 250, such as along pivot pins 215, as illustrated in FIG. 5 and FIG. 6. In some aspects, the gears 214 are mounted on a distal end 250d of the mount 250. In some aspects, the pivot pins 215 are mounted generally axially along the mount 250.

In the example of an embodiment illustrated in FIG. 5 and FIG. 6, the actuator 220 includes a translatable component 222 axially translatable through a sheath 224. The distal end 222d of the translatable component 222 is coupled to one of the tissue separators 212 to move with translation of the translatable component 222. For instance, the translatable component 222 may be pivotably coupled to the proximal end 212p of one of the tissue separators 212, such as along a pivot pin 213. In some aspects, the translatable component 222 is coupled to the gear 214 formed along the proximal end 212p of one of the tissue separators 212. In some aspects, the translatable component 222 is coupled to the tissue separator 212 (e.g., to a gear 214 along the proximal end 212p of the tissue separator 212) via a coupler 226 (such as, without limitation, a clevis or a hinge). In some aspects, the proximal end 226p of the coupler 226 is axially fixed with respect to the distal end 222d of the translatable component 222. Additionally or alternatively, in some aspects, the distal end 226d of the coupler 226 is pivotably coupled with the tissue separator 212. As may appreciated, axial translation of the translatable component 222 moves (e.g., rotates, pivots, shifts, etc.) the gear 214 to which the translatable component 222 is coupled to move the associated tissue separators 212. As may be appreciated movement of a gear 214 of one of the tissue separators 212 causes movement of the gear 214 of the other of the tissue separators 212, so that axial translation of the translatable component 222 thereby causes both tissue separators 212 to move. Accordingly, the actuator 220 may be operably coupled with only one of the tissue separators 212 to move the one of the tissue separators 212 and thereby to cause movement of the other tissue separators 212.

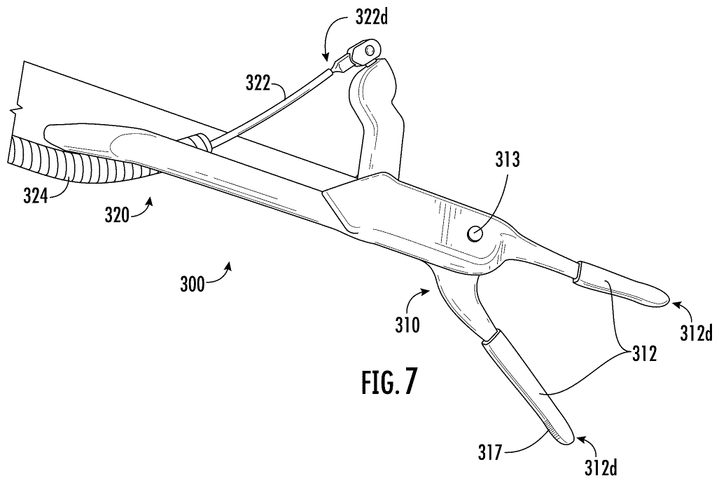

It will be appreciated that principles of the present disclosure may be applied to other configurations of tissue separator devices with pivotably coupled tissue separators. For instance, a tissue-separating system 300 formed in accordance with various principles of the present disclosure may have a tissue separator device 310 with tissue separators 312 simply pivotably coupled about a pivot pin 313, such as illustrated in FIG. 7. An actuator 320 may be coupled to a proximal end 312p of one of the tissue separators 312 to move the one of the tissue separators 312 with respect to the other of the tissue separators 312. The distal ends 312d of the tissue separators 312 may thereby be shifted between a delivery configuration, in which the distal ends 312d are adjacent to each other, and a tissue-separating configuration, in which the distal end 312d are separated from each other, such as described above with respect to the tissue separator device 110 and the tissue separator device 210. Similar to the actuator 220 described above with reference to FIG. 5 and FIG. 6, the actuator 320 may include a translatable component 322 axially translatable through a sheath 324 and having a distal end 322d coupled (e.g., pivotably coupled) to one of the tissue separators 312 to move the one of the tissue separators 312 with respect to the other of the tissue separators 312 with translation (e.g., axial translation) of the translatable component 322. Movement of the tissue separators 312 will cause separation of tissue contacted by the tissue separators 312. In some aspects, microserrations 317 may be formed along the outwardly-facing surfaces the outer sides of the tissue separators 312 (surfaces of each tissue separator 312 facing away from the other tissue separator 312) to facilitate separation of tissue contacting by the moving tissue separators 312. It will be appreciated that the tissue separator device 310 illustrated in FIG. 7 may be mounted with respect to a medical scope, similar to the manner in which the tissue separator device 210 illustrated in FIG. 4 is mounted with respect to medical scope 202.

It will be appreciated that any of the tissue-separating systems described herein may be delivered transluminally within a patient's body with the use of any of a variety of medical devices for navigating body passages or body lumens, including, for example, and without limitation, catheters, delivery tubes, medical scopes (e.g., endoscopes, ureteroscopes, bronchoscopes, colonoscopes, duodenoscopes, arthroscopes, cystoscopes, hysteroscopes, etc.) and the like. For instance, a tissue separator device formed in accordance with various principles of the present disclosure may be delivered through a working channel of a medical scope. In some aspects, a tissue separator device formed in accordance with various principles of the present disclosure may be delivered alongside and/or over a medical scope, the present disclosure not being limited in this regard. The disclosed medical devices and systems may also be inserted via different access points and approaches, e.g., percutaneously, endoscopically, laparoscopically, or combinations thereof.

In view of the above, it should be understood that the various embodiments illustrated in the figures have several separate and independent features, which each, at least alone, has unique benefits which are desirable for, yet not critical to, the presently disclosed examples of an embodiments of tissue separating devices, systems, and methods. Therefore, the various separate features described herein need not all be present in order to achieve at least some of the desired characteristics and/or benefits described herein.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A device for separating tissue, said device comprising:

2. an elongate member having a proximal end and a distal end;

3. a first tissue-separating member having a proximal end operably coupled with the distal end of said elongate member, and a distal end extending distally from the elongate member;

4. a second tissue-separating member having a proximal end operably coupled with the distal end of said elongate member, and a distal end extending distally from the elongate member;

5. a first tissue-separator actuator having a distal end operably coupled with the distal end of said first tissue-separating member; and

6. a second tissue-separator actuator having a distal end operably coupled with the distal end of said first tissue-separating member;

7. wherein said first tissue-separator actuator and said second tissue-separator actuator are shiftable to shift said first tissue-separating member and said second tissue-separating member between a first configuration, in which the distal ends of said first tissue-separating member and said second tissue-separating member are adjacent each other, and a second configuration, in which the distal ends of said first tissue-separating member and said second tissue-separating member are spaced apart from each other.

8. The device of claim 1, wherein when said first tissue-separating member and said second tissue-separating member are in the first configuration, the distal ends of said first tissue-separating member and said second tissue-separating member are at a first axial distance from the proximal ends of said first tissue-separating member and said second tissue-separating member, and when said first tissue-separating member and said second tissue-separating member are in the second configuration, the distal ends of said first actuator and said second actuator are at a second axial distance from the proximal ends of said first tissue-separating member and said second tissue-separating member, the second axial distance being less than the first axial distance.

9. The device of claim 1, wherein said first tissue-separator actuator and said second tissue-separator actuator are shiftable to shift the distal ends of said first tissue-separating member and said second tissue-separating member from the first configuration adjacent each other and at a first axial distance from the proximal ends of said first tissue-separating member and said second tissue-separating member to a configuration in which the distal ends of said first tissue-separator actuator and said second tissue-separator actuator are at a second axial distance from the proximal ends of said first tissue-separating member and said second tissue-separating member, the second axial distance being less than the first axial distance.

10. The device of claim 1, wherein said first tissue-separating member and said second tissue-separating member bend away from each other when shifting from the first position to the second position.

11. The device of claim 1, wherein shifting of said first tissue-separating member and said second tissue-separating member to the second configuration moves tissue contacted by said first tissue-separating member and said second tissue-separating member apart as well as proximally.

12. The device of claim 1, further comprising a control handle and an actuator extending from a proximal end operably associated with said control handle distally to a distal end operably coupled with said first tissue-separator actuator and said second tissue-separator actuator to axially shift said first tissue-separator actuator and said second tissue-separator actuator to shift said first tissue-separating member and said second tissue-separating member between the first configuration and the second configuration.

13. The device of claim 1, wherein:

14. in the first configuration, said first tissue-separating member is adjacent said first tissue-separator actuator and said second tissue-separating member is adjacent said second tissue-separator actuator; and

15. in the second configuration, said first tissue-separating member is spaced apart from said first tissue-separator actuator and said second tissue-separating member is spaced apart from said second tissue separator actuator.

16. The device of claim 1, wherein said first tissue-separating member comprises a pair of tissue-separating members, and said second tissue-separating member comprises a pair of tissue-separating members.

17. The device of claim 1, wherein said first tissue-separating member and said second tissue-separating member are formed from shape memory materials.

18. A device for separating tissue, said device comprising.

19. a first tissue separator having a proximal end and a distal end and comprising a flexible portion and an actuator portion extending between the proximal end and the distal end; and

20. a second tissue separator having a proximal end and a distal end and comprising a flexible portion and an actuator portion extending between the proximal end and the distal end;

21. wherein:

22. said first tissue separator and said second tissue separator are shiftable between a first configuration in which the distal ends of said first tissue separator and said second tissue separator are adjacent each other, and a second configuration in which the distal ends of said first tissue separator and said second tissue separator are apart from each other; and

23. when said first tissue separator and said second tissue separator are shifted to the second configuration, a distance between said flexible portion and said actuator portion of each of said first tissue separator and said second tissue separator increases.

24. The device of claim 10, wherein shifting of said first tissue separator and said second tissue separator from the first configuration to the second configuration increases a tissue separating area defined by said first tissue separator and said second tissue separator.

25. The device of claim 10, wherein shifting of said first tissue separator and said second tissue separator toward the second configuration moves the distal ends of said first tissue separator and said second tissue separator proximally.

26. The device of claim 10, wherein shifting of said first tissue separator and said second tissue separator toward the second configuration causes said flexible portions of said first tissue separator and said second tissue separator to bend away from each other.

27. The device of claim 13, wherein shifting of said first tissue separator and said second tissue separator causes said flexible portions of said first tissue separator and said second tissue separator to flex relative to said actuator portions.

28. The device of claim 14, wherein:

29. each of said flexible portion and said actuator portion of said first tissue separator and said second tissue separator has a proximal end and a distal end;

30. said actuator portion of said first tissue separator has a distal end coupled to distal end of said flexible portion of said first tissue separator;

31. said actuator portion of said second tissue separator has a distal end coupled to a distal end of said flexible portion of said second tissue separator; and

32. said device further comprising an actuator coupled to proximal ends of said actuator portions of said first tissue separator and said second tissue separator to proximally pull said actuator portions to pull the distal ends of said flexible portions proximally to shift said first tissue separator and said second tissue separator toward the second configuration.

33. A method of separating tissue, said method comprising:

34. advancing a tissue separator device to tissue to be separated, the tissue separator device having a first tissue-separating member with a proximal end and a distal end, and a second tissue-separating member with a proximal end and a distal end;

35. inserting the tissue separator device into tissue in a first configuration with the distal ends of the first tissue-separating member and the second tissue-separating member adjacent each other; and

36. shifting the tissue separator device towards a second configuration to move the distal ends of the first tissue-separating member and the second tissue-separating member laterally apart from each other as well as proximally to move the tissue to be separated apart as well as proximally.

37. The method of claim 16, further comprising increasing the surface area of the first tissue-separating member and the second tissue-separating member upon shifting the tissue separator device towards the second configuration.

38. The method of claim 17, further comprising causing a flexible portion of the first tissue-separating member to bend outwardly away from a flexible portion of the second tissue-separating member when shifting the tissue separator device towards the second configuration.

39. The method of claim 16, further comprising pulling an actuator proximally to pull the distal ends of the first tissue-separating member and the second tissue-separating member proximally.

40. The method of claim 19, wherein pulling the distal ends of the first tissue-separating member and the second tissue-separating member proximally causes the distal ends of the first tissue-separating member and the second tissue-separating member to move apart from each other.
